(19) United States
(12) Patent Application Publication
(10) Pub. No.: US 2023/0355988 A1
(43) Pub. Date: Nov. 9, 2023

(54) TRANSVENOUS PACING SYSTEM

(71) Applicants: Puneet Khanna, Rancho Mirage, CA (US); Expanse Technology Partners, LLC, Pleasanton, CA (US)

(72) Inventors: Puneet Khanna, Rancho Mirage, CA (US); Eitan Konstantino, Orinda, CA (US); Matthew D. Ritch, Pleasanton, CA (US); Stephanie M. Boula, Fremont, CA (US)

(21) Appl. No.: 18/041,980

(22) PCT Filed: Aug. 16, 2021
(86) PCT No.: PCT/US2021/071197

§ 371 Date: Feb. 16, 2023

Related U.S. Application Data
(60) Provisional application No. 63/200,044, filed on Feb. 11, 2021.
(60) Provisional application No. 63/066,673, filed on Aug. 17, 2020.

(51) Int. Cl. A61N 1/05; A61N 1/362

(52) U.S. Cl. CPC A61N 1/362; A61N 1/057

(57) ABSTRACT

Temporary pacing catheters for pacing a patient’s heart and methods of use. The pacing catheter may include variable stiffness shaft. A distal portion of the pacing catheter may carry one or more electrodes for pacing the patient’s heart. The pacing catheter may include a lumen configured to receive a shaping instrument. The pacing catheter may have a first configuration when advanced to the patient’s heart without the shaping instrument and a second configuration configured to stabilize the pacing catheter within the patient’s heart when the shaping instrument is introduced through the lumen.

### INCORPORATION BY REFERENCE TO ANY PRIORITY APPLICATIONS

The present application claims priority benefit to U.S. Provisional Application No. 63/066,673, filed Aug. 17, 2020, and U.S. Provisional Application No. 63/200,044, filed Feb. 11, 2021, each of which is hereby incorporated by reference in its entirety herein.

### BACKGROUND

### Filed

The present disclosure relates to the field of medical methods and devices, more specifically to medical temporary transvenous cardiac pacing catheters.

### Description of Related Art

When emergency medical technicians pick up patients in the field with complete heart block, one of their only options in the field is to use an external pacemaker, which requires a high voltage to stimulate the heart. The high voltage is painful for the patient. Often the external pacemaker does not even work because there is not enough current conducted to the heart to stimulate the heart.

When that patient arrives in the emergency room, emergency room physicians attempt to put a temporary pace lead in without visualization (e.g., x-ray or fluoroscopy). But without visualization, there is a risk of perforating the heart and patient death.

### SUMMARY

Temporary transvenous cardiac pacing catheters are one tool for the temporary treatment of advanced heart block as well as for temporary pacing during interventional procedures, profound arrhythmia caused by cardiac block, myocardial infarction, and other medical problems which interfere with the heart’s natural electrical conduction system. These devices are introduced to the heart through peripheral veins and guided to the heart through the superior or inferior vena cava. The catheters typically have exposed electrodes on their distal ends, which are brought into electrical contact with the inside of the heart. Oscillating electrical potentials with amplitudes between 1 and 5 Volts and frequencies between 0.5 and 3 Hertz are then applied to one or both of the electrodes. These potentials cause electrical current to flow between the electrodes through the cardiac tissue, depolarizing the muscle and inducing mechanical contraction of the heart.

To successfully conduct temporary transvenous pacing, a pacing catheter should be inserted into the veins, navigated from the insertion point to the heart, maneuvered into a stable orientation within the heart, and fixated in that position to ensure good electrical capture. The currently available devices and methods for temporary transvenous pacing are not well suited for emergency use because the strategies they employ to complete these requisite steps are not feasible in an emergency setting.

First, most temporary pacing catheters are inserted directly into the subclavian, jugular, or femoral veins through the neck. Inserting a catheter in any of these locations is difficult and potentially hazardous to the patient, requiring experienced physician use and the help of ultrasound equipment. These resources may not be available in emergency situations requiring emergency cardiac pacing, causing a delay in treatment which may lead to injury or death (significant morbidity and mortality).

Second, the standard of care is to use fluoroscopy to navigate the catheter from the insertion point to the heart. Fluoroscopy has many drawbacks. Fluoroscopy exposes both the patient and caregivers to radiation. Additionally, fluoroscopic navigation requires the use of fluoroscopic machines, which are often unavailable in emergency situations. Relying on fluoroscopic navigation in an emergency situation harms the patient and delays their treatment.

Third, the standard of care is to maneuver the pacing catheter so that the electrodes are situated in the apex of the right ventricle. This orientation is fairly stable when the patient is immobile and laying in a bed. However, in an emergency situation, the patient may need to move or be moved in a way that will dislodge the electrodes from the apex, resulting in a loss of capture and injury or death. Additionally, pacing from the apex of the right ventricle causes the ventricles to depolarize and contract opposite to the physiological pattern.

Fourth, the standard of care is to either not to fix the catheter in the proper orientation within the heart or fix it with traumatic mechanisms such as screws. Unfixed pacing catheters have a large risk of dislodging and losing capture, as stated above. Screw fixation catheters are less likely to dislodge, but they cause trauma to the heart tissue. Both with and without fixation mechanisms, existing pacing catheters can cause ventricle perforations, which can result in pericardial tamponade and death, necessitating emergency pericardiocentesis or even thoracotomy and cause permanent injury or death. In an emergency, patient motion will dislodge unfixed electrodes from the right ventricle apex and traumatic screw fixation mechanisms may cause severe damage to a patient’s heart, ventricular perforation.

For these reasons, the systems described herein provide improved temporary transvenous cardiac pacing catheters. The transvenous pacing catheters described herein may be delivered without imaging and without the risk of right ventricular perforation. The pacing catheter may be sufficiently soft (e.g. type or thickness of material) that it will not perforate the chamber when it comes into the right atrium/right ventricle. The pacing catheter can be oriented to pace the right ventricle outflow tract. Once the pacing catheter is in position, the pacing catheter provides a stable and less traumatic form of fixation to ensure good capture. The size of the pacing catheter does not interfere with imaging capability required for adjunctive procedures. The pacing catheter may allow complete mobility of the patient. The pacing catheter may be easily removed without risk of damage to the right ventricle/right atrium. In some configurations, the pacing catheter may be a bipolar system, but in other configurations, may be a quadripolar system that allows atrial and ventricular pacing. The pacing catheter may be small enough to not impede venous flow and cause venous thrombosis. The pacing catheter may measure the distance from the antecubital vein to the superior vena cava from the outside of the patient to estimate (within a few cm) the length of the delivery catheter required for different size patients.

Certain aspects of the disclosure are directed toward methods of delivering a pacing device to a heart of a patient. The method may include advancing a pacing catheter to the heart, for example the right ventricle outflow tract, in a first configuration. The pacing catheter may be delivered over a guidewire or have sufficient pushability alone or in combination with a delivery stylet to be delivered without a guidewire.

The method may include shaping the pacing catheter by advancing a shaping instrument through a lumen of the pacing catheter and causing the pacing catheter to transition from the first configuration to a second configuration. The shaping instrument may be selected from a plurality of different shaping instruments that may be different-shaped and/or different sized. Any one of the plurality of different shaping instruments may be used with the same pacing catheter. The second configuration may have a distal portion shaped to contact a wall of the heart and stabilize the pacing catheter in the heart. After pacing, the method may include removing the shaping instrument from the lumen of the pacing catheter and causing the pacing catheter to transition from the second configuration to the first configuration. The pacing catheter may be removed from the patient in the first configuration.

To facilitate guidance, the method may include transmitting light from or through a sidewall and/or distal tip of the pacing catheter while advancing the pacing catheter to the heart. The transmitted light being visible outside the patient by the naked eye without imaging equipment. The method may include inflating a balloon on the pacing catheter or guidewire to carry the pacing catheter with blood flow.

To facilitate fixation, the method may include deploying a fixation element to stabilize the pacing catheter in the heart without anchoring in the heart wall. The fixation element may be deployed from a sidewall of the pacing catheter. The method may include deploying the fixation element in an atrium or right ventricle of the heart. In some methods, an electrical pulse may be from the fixation element.

Certain methods for pacing the ventricles without visualization are disclosed. The method may include advancing a pacing catheter through the vasculature, for example by inserting the pacing catheter through an IV access. The clinician may advance the pacing catheter using lighting elements on the pacing catheter that are visible from outside the patient by the naked eye without visualization equipment. The method may include guiding a distal end of the catheter body to a right ventricular outflow tract using a flotation balloon on the pacing catheter or guidewire. The method may include stabilizing a position of the catheter by deploying a fixation element in the right ventricular outflow tract. The fixation element may be an atraumatic structure deployed from a sidewall of the pacing catheter.

Certain aspects of the disclosure are directed toward a temporary pacing catheter for pacing a patient’s heart. The pacing catheter may include a variable stiffness, for example the pacing catheter may include a proximal portion having a first stiffness and a distal portion having a second stiffness. Additional variations in stiffness may be provided between the proximal portion and the distal portion. The distal portion may include one or more electrodes to pace the heart. The distal portion may extend less than or equal to about 20 cm, less than or equal to about 15 cm, or less than or equal to about 10 cm from a distal tip of the pacing catheter.

In certain aspects, the pacing catheter may include one or more lumens. The pacing catheter may include a first lumen to receive a shaping instrument. The same or different lumen may be used to advance the pacing catheter over a guidewire. The first lumen may be sealed or open at a distal tip of the pacing catheter. The pacing catheter may include a first configuration when advanced to the patient’s heart without the shaping instrument and a second configuration configured to stabilize the pacing catheter within the patient’s heart when the shaping instrument is introduced through the lumen. A shape of the proximal portion may remain unchanged between the first configuration and the second configuration. In the second configuration, the distal portion may have a spiral shape to contact a wall of the patient’s heart circumferentially or an S-shape to contact a wall of the patient’s heart at different axial locations without circumferentially contacting the wall.

Certain aspects of the disclosure are directed toward a pacing catheter having a catheter body comprising an electrode region. The pacing catheter may include a flotation balloon. The flotation balloon may be positioned in the distal portion of the catheter, for example between the distal electrode and the proximal electrode or at the distal tip. The flotation balloon may be shaped to guide a distal tip of the catheter with blood flow. The pacing catheter may include an inflation lumen configured to supply gas or fluid to the flotation balloon. In other configurations, the guidewire may carry the flotation balloon.

Certain aspects of the disclosure are directed toward pacing catheter having a catheter body comprising an electrode region. The pacing catheter may include a fixation element protruding radially from the catheter body. The fixation element may be a wire extending out of a sidewall of the catheter body. For example, there may be a port or other opening in the sidewall from which the wire fixation element may be released. When released, the wire fixation element may have a loop shape.

Certain aspects of the disclosure may be directed toward a pacing catheter having a catheter body comprising an electrode region. The pacing catheter may include a plurality of lighting elements spaced apart along a length of the catheter body. The one or more light emitting elements may be in the distal portion of the catheter. For example, one or more lighting elements may be embedded in or provided on an exterior surface of the distal portion. In some configurations, the plurality of light sources configured to emit light between 400 and 700 nm. The plurality of light sources may be configured to provide varied intensities or frequencies of light depending on their position along the catheter body.

Any of the pacing catheters described herein may be provided in a kit. The kit may include one or more shaping instruments, e.g., a stylet. The one or more shaping instruments may differ in size and/or shape to cause the pacing catheter to form different shapes in the second configuration. The kit may include a guidewire to advance the pacing catheter to the heart. The guidewire may include a flotation balloon to carry the guidewire and/or the pacing catheter to the target location. The kit may include an optical fiber that may be delivered through the lumen of the pacing catheter. Light from the optical fiber within the lumen may be visible outside of the patient’s body.

Certain aspects of the disclosure are directed toward a dispenser for dispensing a pacing catheter in a sterile manner. The dispenser may also be included in the kit. The dispenser may include a housing for carrying the pacing catheter in a compact configuration, for example in a spiral shape and an outlet for dispensing the pacing catheter from the housing. The dispenser may include a sterile barrier configured to form a seal against the pacing catheter as the pacing catheter is dispensed from the outlet. One or more user controls may be provided to control dispensation of the pacing catheter. The dispenser may include one or more ports for providing power or inflation medium to the pacing catheter.

Any feature, structure, or step disclosed herein can be replaced with or combined with any other feature, structure, or step disclosed herein, or omitted. Further, for purposes of summarizing the disclosure, certain aspects, advantages, and features of the inventions have been described herein. It is to be understood that not necessarily any or all such advantages are achieved in accordance with any particular embodiment of the inventions disclosed herein. No individual aspects of this disclosure are essential or indispensable.

### BRIEF DESCRIPTION OF THE DRAWINGS

FIG. 1 shows a schematic view of a pacing catheter system.

FIG. 2 shows a schematic of a pacing catheter with a fixation element deployed in the right ventricle outflow tract.

FIG. 3A illustrates a distal region of a pacing catheter having a light source integrated into the catheter shaft.

FIGS. 3B to 3C show two possible cross-sections of the catheter shown in FIG. 3A.

FIGS. 4A to 4D show example shapes for a flotation balloon near the distal tip of a pacing catheter.

FIGS. 5A to 5F show example fixation elements that may be used with the catheter shown in FIG. 2A.

FIG. 6 illustrates an exemplary sterile catheter dispenser.

FIG. 7A illustrates a catheter deployed in the right ventricle outflow tract in a first configuration.

FIG. 7B illustrates the catheter shown in FIG. 7A deployed in the right ventricle outflow tract in a second configuration.

FIG. 8A illustrates another catheter deployed in the right ventricle outflow tract in a first configuration.

FIG. 8B illustrates the catheter shown in FIG. 8B deployed in the right ventricle outflow tract in a second configuration.

Various embodiments are depicted in the accompanying drawings for illustrative purposes, and should in no way be interpreted as limiting the scope of the embodiments. Furthermore, various features of different disclosed embodiments can be combined to form additional embodiments, which are part of this disclosure.

### DETAILED DESCRIPTION

The present disclosure provides methods and devices for emergency treatment of advanced heart block or transvenous pacing during interventional procedures ventricular arrhythmias through transvenous temporary pacing without relying on resources that are frequently unavailable in an emergency. The transvenous pacing catheters are designed to be safely and quickly used in emergencies or other situations lacking the resources usually necessary for temporary transvenous pacing, including but not limited to ultrasound devices, fluoroscopy machines, and experienced cardiologists.

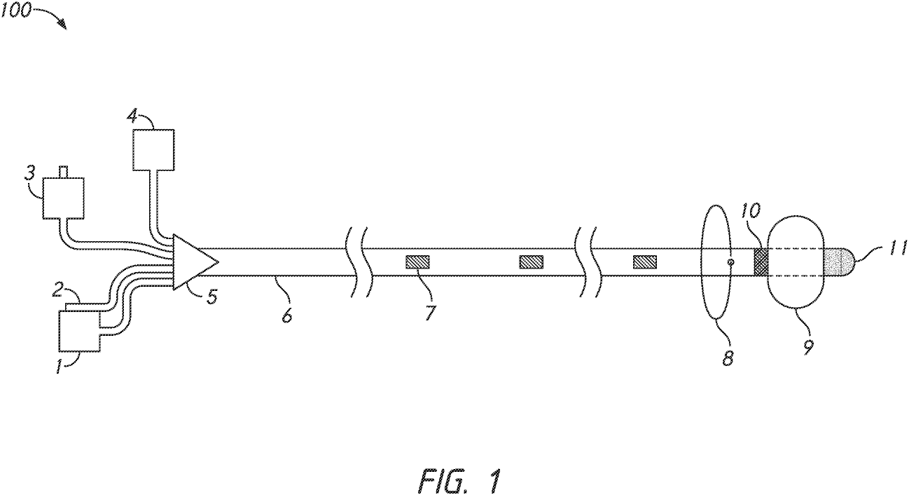

FIG. 1 illustrates a schematic pacing catheter system that may include a distal 1 and/or proximal 2 safety power jack for a standard external temporary pacing box used to power distal 11 and/or proximal 10 electrodes at the distal portion of the catheter 100. The distal electrode 11 may be at or adjacent a distal tip of the catheter 100. The electrodes 10, 11 may be adhered or otherwise joined to an external surface of the catheter 100. Current may be delivered to the electrodes 10, 11 through a conductor embedded in the walls of the shaft 6 or a dedicated lumen of the catheter 100. Additional electrodes may be provided.

A luer lock 3 may be provided to inflate a flotation balloon 9 at the distal portion of the catheter 100, for example the flotation balloon 9 may be provided between the electrodes 10, 11. In other configurations, the flotation balloon 9 may be provided proximal of the proximal electrode 10 or distal of the distal electrode 11. The system may include a plug-in 4 for powering or supplying light to the light sources 7 embedded along or visible through the shaft 6. The system may include a cable joiner region 5 for the different powered components. The system may include a fixation element 8, such as a wire loop, to stabilize a position of the pacing catheter without necessarily anchoring in the tissue. The fixation element 8 may be released from a sidewall of the catheter.

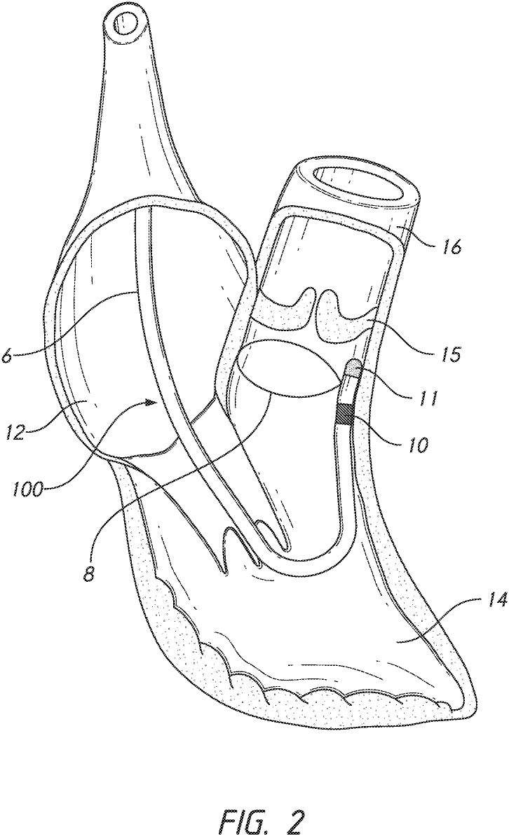

As shown in FIG. 2, the pacing catheter 100 may be deployed through the right atrium 12, through the right atrioventricular valve 13, into the right ventricle 14, and into the right ventricle outflow tract before or near the pulmonary valve 15 and trunk 16.

Any of the pacing systems described herein may include features for maintaining the catheter’s sterility while it is being inserted into the veins, even when the pacing catheter is used to treat a patient outside of traditional clinical environments. As shown in FIG. 1, the pacing system may include navigational lights 7, which change their intensities and/or frequencies with their anatomical location, providing signals which indicate the catheter’s progression through the body to the user. The pacing system may include a flotation balloon 9 having geometries that enable the user to place the pacing electrodes within the right ventricle outflow tract instead of the right ventricular apex. The pacing system may include a fixation element 8 that enables the user to stabilize or fix the pacing electrodes 10, 11 within the right ventricle outflow tract instead of the right ventricular apex. Any one or more of these features may be provided alone or different combined to form different embodiments.

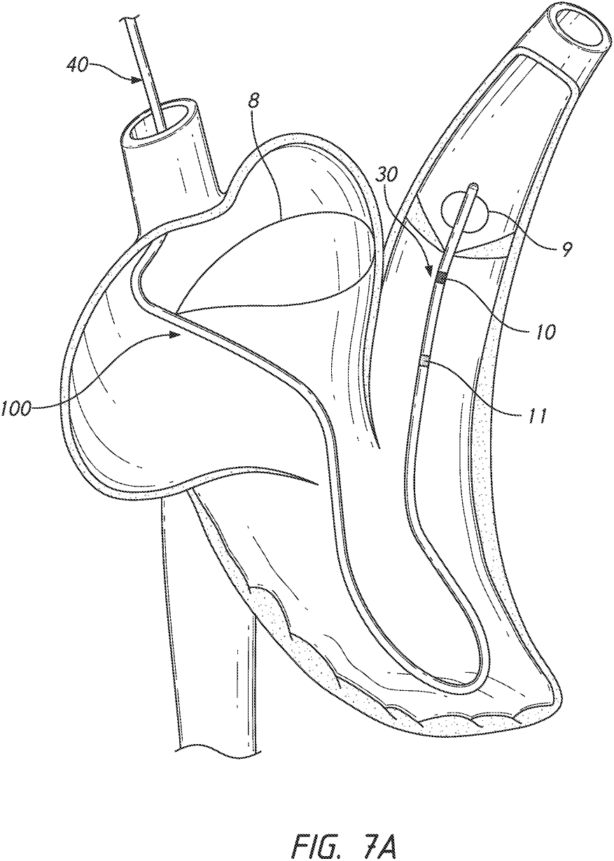

As explained in further detail below, the catheter 100 may be inserted into the vein from a sterile dispenser device 150 (see FIG. 6) and/or through a sterile shield. These mechanisms protect the sterility of the catheter 100 even when it is being inserted with a limited sterile field or outside of a traditional clinical context.

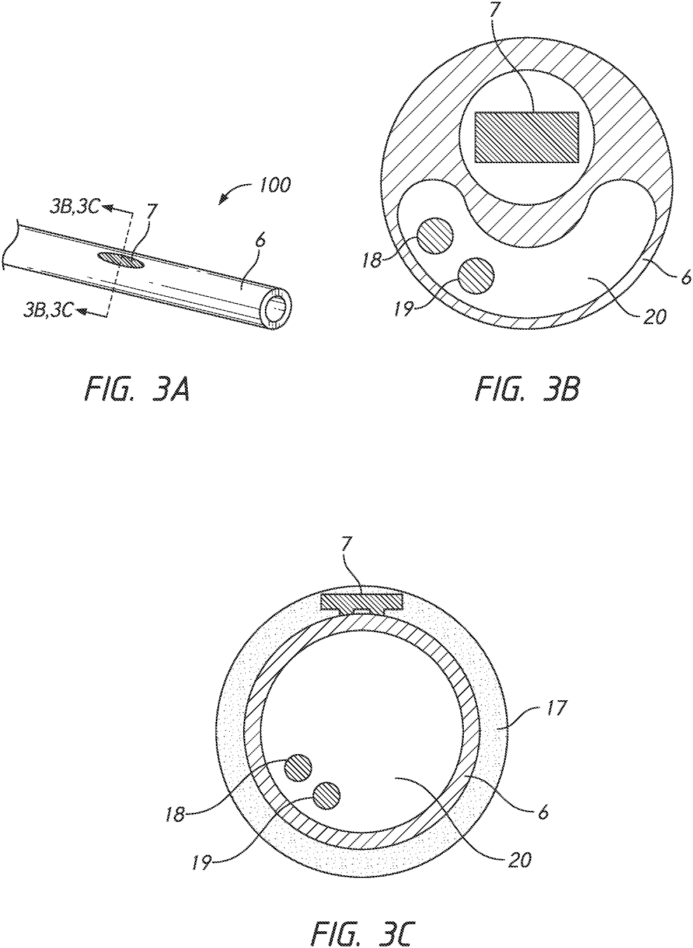

Once the user has inserted the pacing catheter 100 into a vein, the user must navigate the pacing catheter 100 from the insertion point to the right ventricle 14 of the heart. In some embodiments, the pacing catheter 100 may include light sources 7 embedded along or visible through the outside of the catheter shaft 6 that transmit light outside of the patient (see FIGS. 3A-3C). This light may be visible to the user’s naked eye, enabling navigation without fluoroscopy. These light sources 7 may change their frequencies or intensities in response to their anatomical context, providing signals to the user. These signals help the user know where the catheter is and where it is going within the body. Navigational lighting as described herein can be particularly important, for example in an antecubital approach, where it is possible the guidewire or catheter to be advanced toward the neck instead of the heart when no imaging is available.

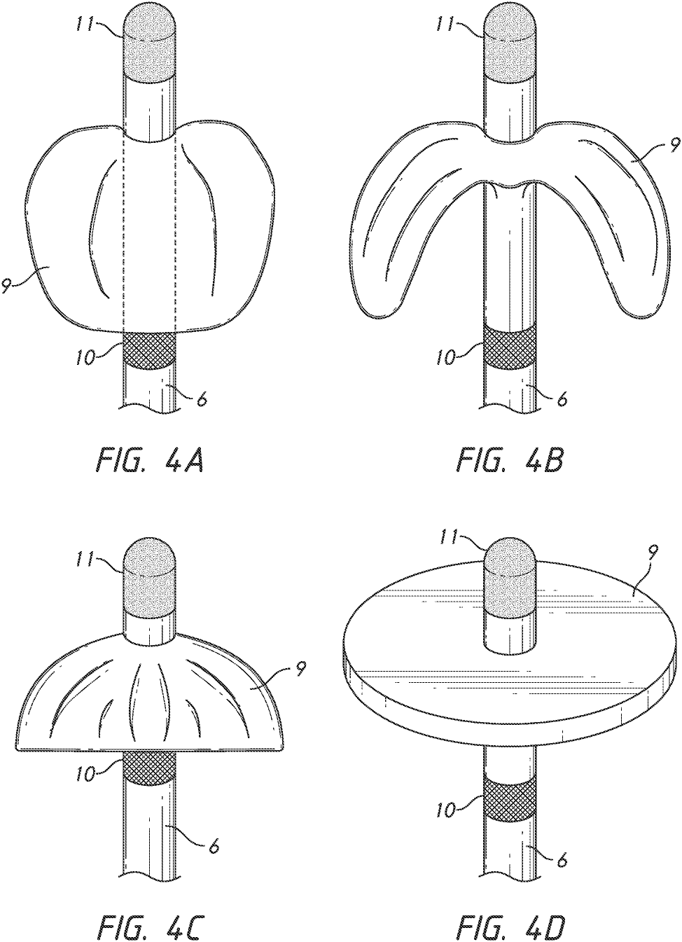

Once the pacing catheter 100 reaches the right ventricle 14, the user must be able to orient its tip properly within the right ventricle outflow tract. The pacing catheter 100 may include a balloon flotation component 9 (see FIGS. 4A-4D), which is designed to enable the user to orient the tip of the catheter 100 within the right ventricular outflow tract so that the proximal and distal electrodes 10, 11 are pacing through the right ventricular septum. The balloon 9, once deployed, follows the flow of blood and induces the tip of the catheter 100 to deflect towards its proper orientation against the ventricular septum within the right ventricular outflow tract. The balloon geometries may be specifically designed to fit within and stabilize the catheter 100 within the right ventricle outflow tract, for example until the fixation element 8 can be activated. Since the balloon 9 is carried with local blood flow, the balloon 9 provides an atraumatic method to carry the pacing catheter 100 to the right ventricular outflow tract without visualization.

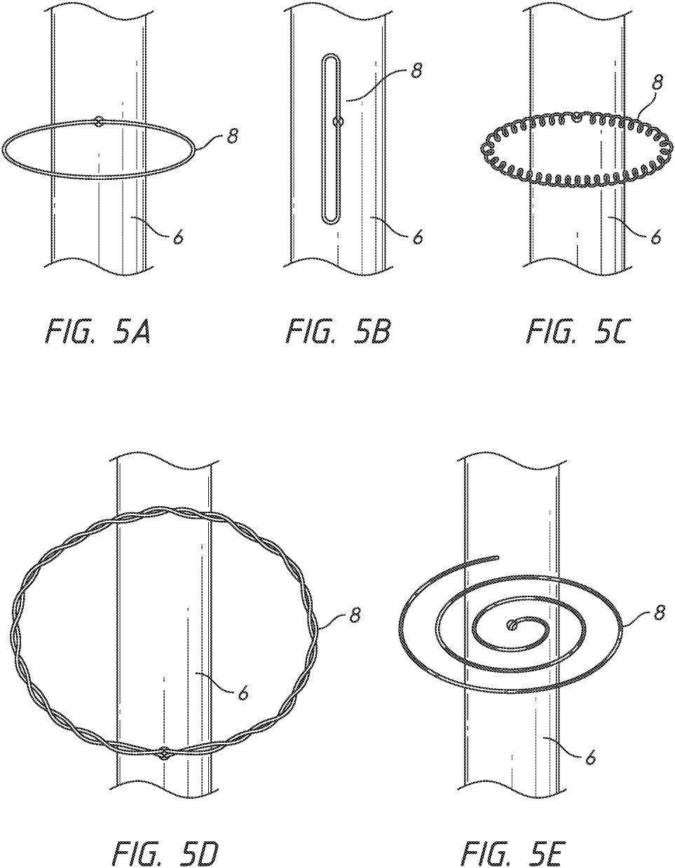

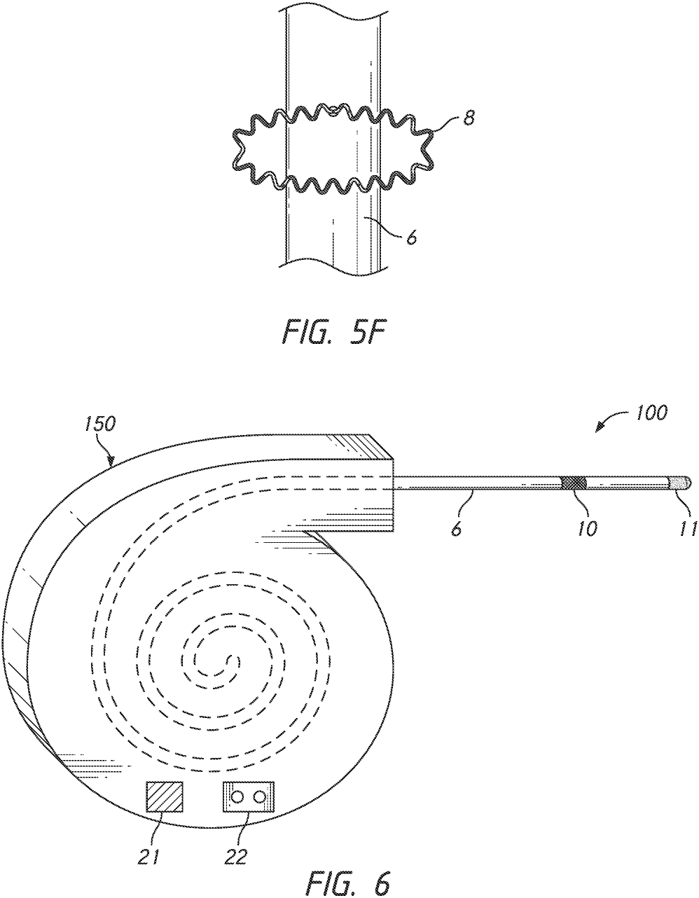

Once the pacing catheter 100 is properly oriented within the right ventricle 14, the catheter may be able to retain that orientation. The catheter 100 may include at least one fixation element 8, which may deploy from a sidewall of the distal portion of the catheter 100 (or elsewhere along the catheter shaft 6) to stabilize or fix the catheter 100 within the right ventricular outflow tract and thereby ensure good electrical contact between the electrodes 10, 11 and the cardiac tissue (see FIG. 2). The fixation element 8 may be able to maintain this contact even while the patient walks, sits, lays down, or is carried. The fixation element 8 may be specifically designed to stabilize or fix the catheter within the right ventricle outflow tract (see FIGS. 5A-5E).

The pacing catheters described herein may be delivered without imaging. For example, the pacing catheters described herein can be inserted into the vein without ultrasound and may be inserted through an existing IV port. Inserting a pacing catheter under ultrasound requires equipment and expertise which may not be available in an emergency situation. Emergency medical professionals are familiar with inserting and using IV ports, so the systems described herein enable them to apply cardiac pacing safely and quickly in emergencies.

Since it is likely that the emergency medical technician or other clinician will start an IV, the IV access can be used to deliver the pacing catheters. For example, the procedure may be initiated by placing an 18 gauge Angiocath in the antecubital vein. The Angiocath may be used to introduce a 0.035 in J-wire. The Angiocath may be removed and the wire may be used to insert a 6 or 5 French side-arm sheath. IV fluids may be attached to the side arm sheath to allow infusion of fluids, which help distend the vein and facilitate delivery of the transvenous pacing system.

Other example approaches for delivering the pacing system include, but are not limited to, standard internal jugular or subclavian venous access with a 6 French sheath; or 6 French sheath in the antecubital vein delivered over a 0.035 wire through an 18 gauge Angiocath initially placed in the cephalic vein.

### Navigational Guidance

Referring now to FIGS. 3A through 3C, two options for embedding the light sources 7 within the catheter shaft 6 are shown. FIG. 3B shows the light source 7 placed within a lumen inside the shaft wall so that it is isolated from the other lumens, such as an inflation lumen 20 for the flotation balloon 9. FIG. 3C shows the light source 7 placed on top of the shaft wall and held in place, for example with a laminate 17. Both cross sections show the distal 18 and proximal 19 electrode wires running through a separate lumen.

In some embodiments, the catheter 100 may include one or more light sources 7 spaced along its external surface or embedded within the shaft wall. The light sources 7 are configured to be visible to the naked eye outside the patient’s body. The light sources 7 are configured to provide visual reference of the catheter’s 100 current position to the clinician. The light sources 7 may be powered by, or the light may be transmitted through a braid and/or a coil, which is placed into the catheter wall and will also be used for catheter reinforcement. The light sources may be LEDs, lasers, light scattering or emitting optic fibers, or some combination of these.

The light sources 7 may be spaced in a way such that their relative intensities indicate the catheter’s location relative to landmarks such as the clavicle. For instance, there may be a light at or near the catheter tip to indicate where the catheter 100 is heading. This light disappears when the catheter 100 crosses into the subclavian, as the clavicle bone does not allow the transmission of the light. This indicates to the user when the tip of the catheter is approaching the vena cava. This light also indicates to the user if the catheter 100 has entered an improper branch, such as if it has turned around and entered a descending path from an ascending path, such as entering the basilic vein in a descending direction from the cephalic vein in an ascending direction, or if the catheter has entered the internal jugular vein instead of the brachiocephalic vein.

There may also be a light 7 positioned between 10 and 25 cm, for example between 17 and 23 cm, or between 20 and 23 cm from the tip. Because the portion of the subclavian which is under the clavicle is about this length of vein from the heart, this light 7 will be obstructed by the clavicle when the tip is most likely within the right ventricle of the heart. This reduction of the light’s transmitted intensity signals to the user that they should deploy the flotation balloon and the fixation mechanism. Other lights may be spaced at even intervals along the length of the catheter shaft, allowing the user to measure the total length of catheter which they have inserted.

The frequencies of the emitted light may be between 400 and 700 nm, for example between 580 and 670 nm, or between 620 and 660 nm. The red spectrum allows for the most favorable combination of low light absorption and low scattering in muscle, skin, and fat, allowing the light to shine through the tissue enough to be seen by the user. Each light source’s intensity may be within 1 and 4 lumens, for example within 2 and 3.5 lumens, or between 2.5 and 3 lumens. These ranges allow for maximum transmitted light intensity without generating dangerous levels of heat within the body. The intensities or frequencies of light emitted may also oscillate or vary depending on their position along the catheter or on the catheter’s orientation within the body. For instance, the intensities of lights placed towards the distal tip of the catheter may oscillate with higher frequencies than lights placed towards the proximal end of the catheter. This allows the user to observe the catheter’s orientation as it varies along its length. Higher frequency intensity oscillations may also be easier for the user to perceive the light emanating through the patient’s tissue.

Simultaneously, an electrical signal from the tip of the pacing device may be sensed to allow for confirmation of positioning in the right ventricle.

In other configurations, an optical fiber or LED light may be placed at or on the tip of the catheter, which allows visualization of where the tip of the catheter is as it is advanced through the venous system. In some configurations, a laser emitting fiber may be tracked with an external sensor, or a sound limiting fiber may be tracked with sonographic equipment. In other configurations, an optical fiber may be advanced through a lumen of the catheter and visible through the catheter wall and/or at the catheter tip.

In some embodiments, the catheter 100 may include pressure sensors distributed along its length and around the tip. These pressure sensors may control indicators that display themselves to the user when they are navigating in the proper direction or the improper direction, or when they have properly or improperly oriented the catheter within the heart.

To facilitate guidance of the pacing catheter to the desired position, an external magnet may be used to help move the pacing catheter to its desired location in the right ventricle. For example, an external magnet placed to the left of the sternum to help affix the pacing device to the right ventricular wall.

### Flotation Balloon

FIGS. 4A through 4D show various flotation balloon designs. In some embodiments, the catheter 100 may include a flotation balloon 9. The balloon 9 may be positioned between the proximal and distal electrodes 10, 11 and configured to inflate radially outward from the catheter shaft 6. This balloon 9 may be configured to follow the local flow of blood when expanded, causing the tip of the catheter to direct itself with blood flow. The balloon 9 may be configured to enable the user to orient the catheter 100 within the right ventricular outflow tract so that the patient’s heart can be paced from the ventricular septum. Pacing the ventricles of the heart from the right ventricle outflow tract and the ventricular septum is safer and more consistent with normal cardiac conductive function than pacing from the right ventricular apex.

As shown in FIG. 4A, the balloon 9 may be bulbous or ball-shaped. The balloon 9 may be able to accommodate up to 1 cc or up to 1.5 cc of fluid. The balloon 9 may have an outer dimension between 0.5 and 3 cm, for example between 1 and 1.5 cm. The balloon 9 may be designed to better track the local blood flow, such as being shaped like an umbrella (FIG. 4C), a plate FIG. 4D), or wings (FIG. 4B). As shown in FIG. 4C, the balloon shape may be a hemisphere, with one side curved (e.g., distal side) and the other side approximately flat (e.g., proximal side). The balloon 9 may also be shaped like an umbrella, in which case the balloon thickness between its topside and underside may be relatively constant between 0.1 and 1 cm, for example between 0.4 and 0.5 cm. As shown in FIG. 4D, the balloon 9 may also be plate or plane shaped. In this case, the balloon 9 may be shaped like a short cylinder or a prism. The prism’s end polygons may have between 3 and 10 sides, for example between 3 and 5 sides. The height of the cylinder or prism may be between 0.1 and 1 cm, for example between 0.4 and 0.5 cm. The balloon 9 may also be shaped to hold the catheter in its proper orientation within the right ventricle until the fixation element(s) 8 may be deployed. For example, the balloon 9 may be donut shaped with an inner radius between 1.0 and 1.5 cm, enough to allow blood to continue flowing through the outflow tract and the pulmonary valve, and an outer radius between 1.5 and 2.5 cm to hold the catheter secure within the outflow tract and against the pulmonary valve.

The balloon 9 may be inflated as soon as the catheter tip is in a vessel large enough to accommodate the expanded balloon, for example when the balloon is underneath the clavicle.

### Fixation Element

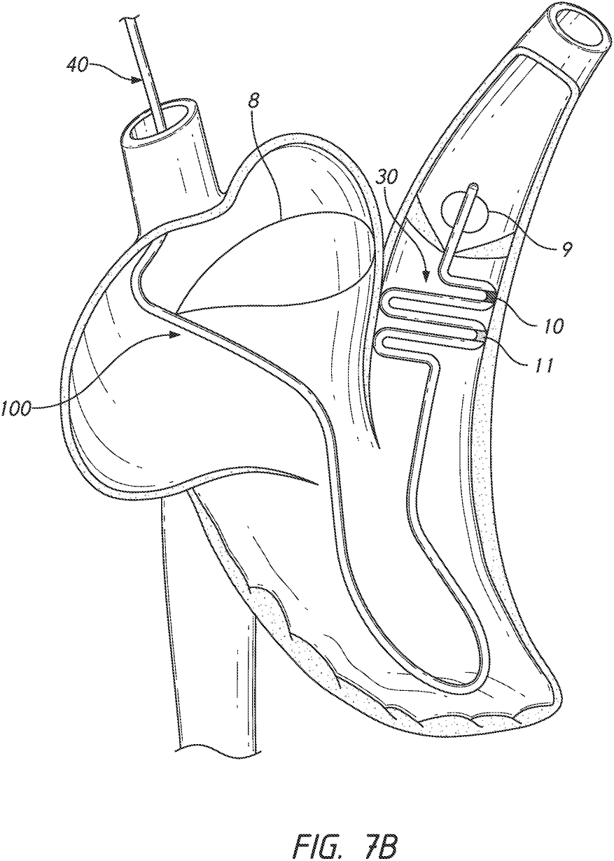

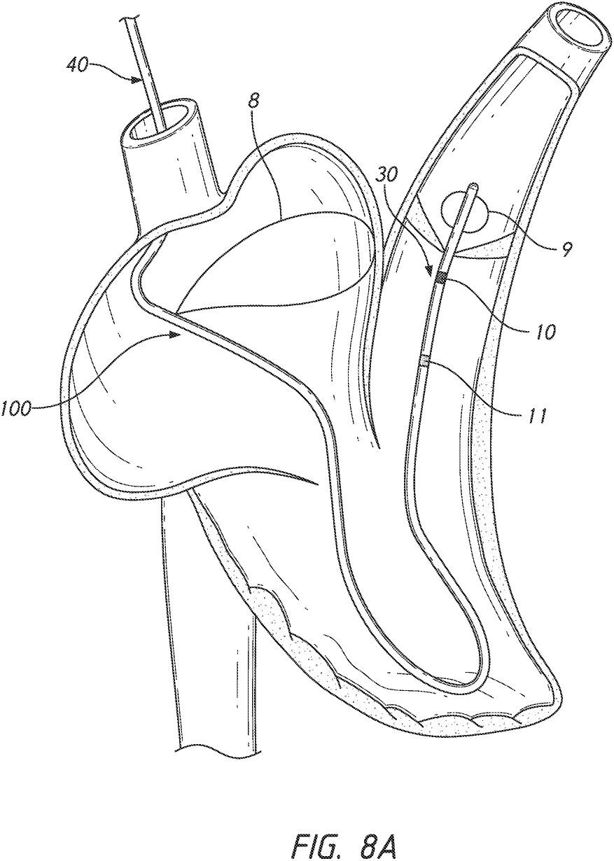

FIGS. 5A through 5F show various fixation element configurations. The fixation elements may be able to stabilize a position of the catheter for pacing without anchoring the catheter in the tissue. In some embodiments, the catheter 100 may include at least one fixation element 8, which protrudes radially from the distal or electrode region of the pacing catheter 100 or elsewhere along the catheter shaft 6 (as shown in FIG. 7B). For example, the fixation element 8 may be released between the proximal and distal electrodes 10, 11. The fixation element 8 may take the shape of a wire loop. Such protruding wire loops enable fixation within the right ventricle outflow tract, ensuring good electrical contact between the electrodes and the cardiac tissue. The fixation element 8 may be configured to resist radial forces of at least 1 N or at least 2 N before they deform to have a greater than 15% change in radius, enabling them to provide good electrical capture for hours or days, even with patient motion. To accomplish this, the wire making up the fixation element 8 may have cross section diameters within the range of 0.004” to 0.014”, for example within the range of 0.007” to 0.010”, or within the range of 0.008” to 0.009”. The wire loops may be oriented with any of the three Cartesian planes around the catheter or in some combination of these orientations. One or many wire loops may be used. These loops may be made of nitinol, stainless steel, or another such biocompatible material.

The overall loops may be circular or elliptical and may have three dimensional aspects. If they are circular (see FIG. 5A), the fixation element 8 may have diameters adapted to fit the size of the anatomy which will typically be between 2 cm to 5 cm, for example between 2.5 cm and 4.0 cm, or between 2.75 cm and 3 cm. If they are elliptical (see FIG. 5B), the fixation element 8 may have a long axis with diameter between 2 cm to 5 cm (or more depending on the anatomical fit), for example between 2.5 cm and 4.0 cm, or between 2.75 cm and 3 cm. The short axis may be of a diameter between 1 cm to 3.5 cm (or more depending on the anatomical fit), for example between 1.5 cm and 3.0 cm, or between 1.75 cm and 2.25 cm. The overall loops may also be three dimensionally shaped like horse shoes, a segment of a spring, 2D or 3D sinusoids, or spirals, or alike (see FIG. 5E). If the loops are shaped like a spring, they may have total height to fit the anatomical requirements, typically between 1.5 cm and 5 cm, for example between 2.5 cm and 3.5 cm, or between 3 cm and 3.25 cm. The spring may have between two and ten coils within this length, for example between four and six coils within this length, such as five coils within this length. If the loops are shaped like sinusoids, they may have periods along their arc length between 1 cm and 5 cm. They may also have amplitudes between 0.5 cm and 1.5 cm. As shown in FIG. 5E, the fixation element may have a spiral configuration with a series of concentric loops, with each loop having a different diameter. All of these shapes are suited to expanding within and taking the shape of the right ventricle outflow tract, allowing proper fixation.

The wires which make up the fixation element 8 may also be locally coiled into a spring shape or patterned along a 2D or 3D sinusoid on in addition to the shape of the overall loop. If the wires are coiled into a local spring shape (see FIG. 5C), they may have a coil radius between 0.012” and 0.040”, for example between 0.016” and 0.022”, and a coil spacing of between 3 and 6 coils per cm of free length, for example between 4 and 5 coils per cm of free length. If the loops are locally shaped like sinusoids, (see FIG. 5F) the local sinusoids may have periods along the loop length between 0.2 and 1 cm, for example between 0.2 and 0.5 cm. The local sinusoids may also have amplitudes between 0.5 and 1.5 cm. These local patterns make the loops more resistant to compressive forces within the right ventricle outflow tract.

These fixation elements 8 may be deployed by advancing a wire into the catheter 100 or by activating a slide mechanism on the side of the catheter. The catheter 100 may include a dedicated lumen for the wire. In some configurations, the wire may be advanced through the catheter 100 in a bent configuration. For example, the wire may be bent and the bent end may be introduced through the lumen. The wire may be released in a loop configuration from an opening in a side wall of the catheter. The size of the loop may be increased by continuing to advance the wire out of the catheter until the loop contacts the heart wall. In some embodiments, the fixation element 8 may include one or more electrodes to provide electrical pulses to the heart wall.

To provide additional fixation, any of the pacing catheters described herein could have tines or retractable hooks that engage in the trabeculations inside the right ventricle. For example, silicone tines or hooks may catch on the trabeculations of the right ventricle. The tines or hooks may only be exposed after reaching the pulmonary artery as this would ensure that the tines or hooks do not catch on the right atrium/tricuspid valve. The tines may be sufficiently small such they may be pulled out without causing any damage. In other configurations, multiple soft wires could be exposed at the tip of the pacing system having barbs. When brought backwards through the right ventricle, the barbs could anchor into the trabeculations. In some embodiments, tendril wires may be released that would entwine the trabeculations of the right ventricle.

Additionally or alternatively, the pacing system may be fixed via a magnetic field with an external magnet. For example, the external magnet could be put on the chest like an adhesive EKG monitoring pad.

### Sterile Barrier

FIG. 6 shows an exemplary system including a sterile catheter dispenser 150 that may include an inflation port 21 for the flotation balloon and/or a power port 22 for the electrode jacks.

In some embodiments, the catheter 100 may be deployed directly from a dispenser 150 designed to keep the catheter sterile in a potentially non-sterile setting. The catheter 100 may come pre-loaded inside the dispenser 150. The dispenser 150 may have an opening that can be closely aligned or joined to an existing IV port or introducer sheath, maintaining catheter sterility during insertions. The dispenser 150 may have manual controls on its outer surface, enabling the user to dispense the catheter without directly touching it. These manual controls may be a crank arm or a roller wheel. The dispenser 150 may also have a powered dispensing mechanism and controls on its outer surface which enable the user to control the dispensing mechanism.

The catheter 100 may be released through a sterile barrier on the dispenser 150. This sterile barrier may comprise a tube containing series of polymer leaflet barriers that may be made of Tyvek or silicone. Each barrier may have between 2 and 6 leaflets, for example 3 or 4 leaflets. These leaflets obstruct the sterile barrier at rest but part with applied pressure to allow the catheter to advance. The leaflets remain in contact with the catheter as it passes, keeping a seal to prevent contamination from entering the dispenser. These barriers may have a diameter between 1.5 and 3 mm, for example between 1.5 and 2.25 mm or between 1.5 and 2.0 mm. The barriers may be spaced within the tube at regular intervals between 0.01” and 0.5” apart, for example between 0.05” and 0.25” apart, or between 0.10” and 0.25” apart. There may be 2 to 10 barriers, for example 2 to 4, or be 3 or 4 of these barriers. This redundancy maintains increased sterility inside the dispenser. The dispenser 150 and the tube of the sterile barrier may use positive pressure or contain disinfectant substances that maintain their sterility while the catheter is being dispensed through the barriers. The dispenser 150 may also display the current length of the catheter 100 that has been dispensed, allowing the user to measure how far they have advanced the catheter within the patient’s body.

### Catheter Shaping

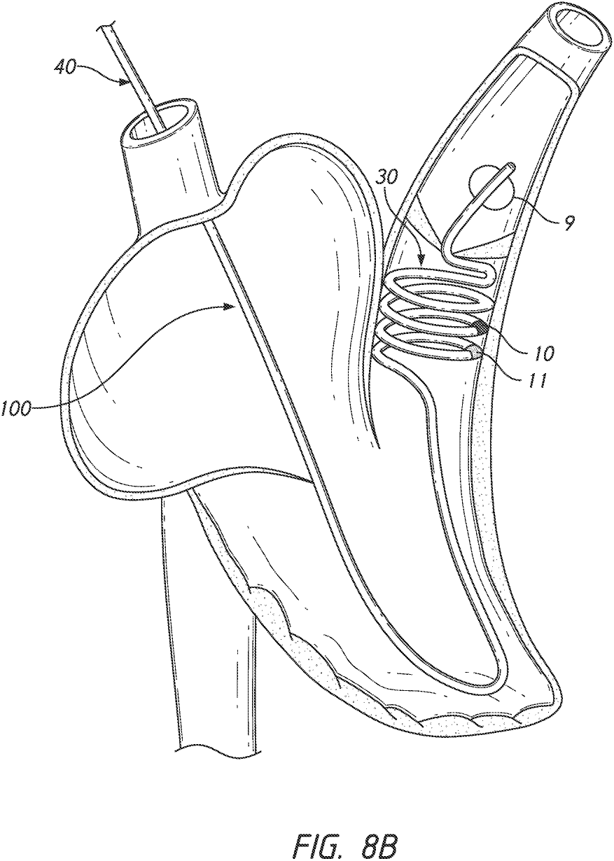

The catheter 100 may be advanced into the pulmonary artery, for example using any of the guidance features described above, in a first configuration (see FIGS. 7A and 8A). A confirmational change may occur in the catheter by virtue of different shaping instruments (e.g., stylet or wire) that can be advanced into the catheter 100. This confirmational change of shape may cause the catheter 100 to transition to a second configuration (see FIGS. 7B and 8B). In the second configuration, at least a distal portion 30 takes the shape of an S curve (see FIG. 7B), shape of a C curve, spiral (see FIG. 8B), or other shape. As shown in FIGS. 7B and 8B, this change allows catheter apposition against the right ventricular outflow tract as well as an element of stabilization or fixation (in an atraumatic fashion) that keeps the catheter position from moving. The distal portion 30 in contact with the heart wall may carry one or more electrodes 10, 11 to provide electrical pulses to the heart. The electrodes 10, 11 may be flexible electrodes, for example in strips or bands, such that any part of the distal portion 30 that comes into contact with the heart wall can deliver electrical pulses to the heart.

FIGS. 7A to 8B illustrate pacing catheters 100 that can change shape. The shape of the pacing catheters 100 may be changed by using shaping elements such as shaping instruments (e.g., wires, mandrels or stylets). The shaping elements can be inserted to the catheter using a dedicated lumen or in some cases using the guidewire lumen. The dedicated lumen may be sealed at its distal end. In other configurations, the guidewire lumen can be used for advancement of shaping elements with a-traumatic tip.

To enable changing the shape of the distal portion 30 of catheter 100 by means of shaping instruments, the catheter 100 may be designed with a variable flexural modulus. The catheter 100 may have a higher modulus and increased resistance to bending at a proximal portion 40 of the catheter 100. The proximal shaft may have a flex modulus of at least 50%, at least 60^, or at least 75% higher than the distal portion. This can be achieved, for example, by using braided or coiled shafts, thicker extrusion wall and/or stiffer materials compared to a distal portion of the catheter. The proximal portion 40 may form a majority of a working length of the catheter 100. The catheter 100 may have a lower resistance to bending at the distal portion 30 of the catheter. This lower resistance makes the distal portion 30 atraumatic to the vasculature. The distal portion 30 may extend no more than 10 cm, no more than 15 cm, or no more than 20 cm from a distal tip of the catheter 100.

Shaping instruments may be pre-shaped to their final configuration with shape recovery properties that cannot overcome the resistance of the proximal portions 40 of the catheter 100. This means that, when the shaping instrument is inserted into the catheter 100, the shaping element will keep a relatively straight configuration in the proximal portion 40, or follow the shape of the proximal portion 40, as long as the shaping instrument is in the high flex modulus area or proximal portion 40. Once the shaping instrument reaches the distal portion 30 of the catheter 100, which is more flimsy than the proximal portion 40, the shaping instrument will reshape the distal portion 30 to assume a two dimensional (see FIG. 7B) or three dimensional configuration (see FIG. 8B) for a traumatic fixation without the risk of perforation. Shaping instruments may be made of metal such as stainless steel or nickel titanium alloys and can also be made from harder polymers typically having shore A hardness greater than 80 or able to be stronger than the distal portion of the catheter.

The pacing catheter 100 may be configured to receive any one of a plurality of different shaping instruments to form a different shaped and/or sized distal portion 30 in the second configuration. The pacing catheter 100 may be provided with the one or more shaping instruments in a kit to allow the clinician to select a shaping instrument to form a suitable shaped or sized distal portion for the patient’s anatomy. The kit may also include any of the other system components described herein.

The catheter 100 may be configured to receive a first shaping instrument to cause the distal portion 30 to transition into a second configuration having an first shape, for example an S-shape (FIG. 7B). In the S-shape configuration, curvatures in the shaped distal portion 30 may contact the tissue at axially spaced apart locations without circumferentially surrounding the heart wall, for example where the electrodes 10, 11 are located in FIG. 7B. The same catheter 100 may receive a second shaping instrument configured to cause the distal portion 30 to transition into a second configuration having a second, different shape, for example a spiral shape (FIG. 8B). In the spiral configuration, the distal portion 30 may be curved to contact circumferential regions of the tissue. Additional shaping instruments may provide yet other shapes, for example to cause the distal portion 30 to form a C-shape.

In other configurations, each of the shaping instruments may form the same shape, e.g., an S-shape or a spiral shape, but in different sizes, e.g., lengths and/or diameters. For example, the first shaping instrument may cause the distal portion 30 to form a shape, e.g., a spiral, having a first outer diameter, and the second shaping instrument may cause the distal portion to form the same shape having a second outer diameter that is different from the first outer diameter. As another example, the first shaping instrument may cause the distal portion 30 to form a shape, e.g., a spiral, having a first axial length, and the second shaping instrument may cause the distal portion to form the same shape having a second axial length that is different from the first axial length. With a spiral shape, the length might change by including different numbers of helical turns or changing the distance between helical turns.

The pacing catheter 100 may be advanced to the heart over an atraumatic guidewire, for example a 0.014 inch guidewire. In some configurations, the guidewire may carry the flotation balloon 9 instead of the catheter shaft 6. The flotation balloon 9 may be used to carry the guidewire to the right ventricle outflow tract prior to advancing the pacing catheter 100 over the guidewire. The flotation balloon 9 may increase the likelihood that the guidewire reaches the target location without visualization. After the pacing catheter 100 has been introduced to the target location, the shaping tool may be introduced into a dedicated lumen or the guidewire may be exchanged with the shaping tool.

In some configurations, the pacing catheter 100 may have sufficient trackability without being advanced over a guidewire, or the pacing catheter 100 may be advanced in combination with a delivery stylet without the use of a guidewire. For example, a delivery stylet may be introduced into the dedicated lumen for the shaping instrument. The delivery stylet may improve the trackability of the distal portion 30 of the catheter 100 for delivery. After the catheter 100 has been advanced to the heart, the delivery stylet may be exchanged with the shaping instrument.

When the pacing catheter 100 needs to be removed, fixation can be reversed by removing the shaping instrument. When the shaping instrument is removed from the pacing catheter 100, the pacing catheter 100 can return to the first configuration and loses the shape in the distal portion 30. This allows the pacing catheter 100 to be atraumatically removed.

Similar mechanisms of fixation and apposition may be used in the atrium, in addition to or in alternative to the right ventricular outflow tract, by either a fixation element 8 as described above or a confirmational change by virtue of different shaped tools. For example, as shown in FIG. 7B, a fixation element 8 may be released from a sidewall of the catheter in the atrium. The fixation element 8 may carry one or more electrodes to deliver electrical pulses. This enables dual chamber pacing in both the atrium and the right ventricle. The timing of the pacing between the two chambers may be adjusted to mimic a physiological heart contraction of both chambers.

Any of the lumens described above, for example the dedicated lumen for the shaping tool or fixation element, may be used to deliver an optical fiber or other light source. Similar to the guiding lights 7 described above, the light emitted from the optical fiber may be visible through at least the distal portion 30 of the catheter wall to facilitate navigation. With optical fibers, the light may appear as a line from outside of the body, which may be more useful than a point light source, because it shows how the catheter 100 is bending within the body. The optical fiber may be exchanged with the shaping tool or a fixation element 8.

### Other Pacing Devices

Additional pacing systems are described below. The pacing systems described below may include any of the features (e.g., guiding or fixation features) of the systems described above. In some configurations, a delivery catheter loaded with a pacing device may be advanced through a sheath. The delivery catheter may have a hydrophilic coating so that it slides easily through the venous system. The delivery catheter may have the ability to detach the pacing device once the catheter is approximately 50 cm in the body. The tip of the catheter that carries the pacing device may have an inflatable balloon, as described above, that allows the device to be advanced into the superior vena cava or even the right atrium.

The pacing device may be pellet-shaped with an atraumatic cord extending back to the pacemaker. The pellet-shaped pacing device may have a generally cylindrical body with rounded ends. The pellet may not have any curvature within the body. A diameter of the pellet may be less than or equal to about 2.0 mm. A length of the pellet may be less than or equal to about 2 cm or less than or equal to about 1 cm. The electrical cord may be very light so it does not impede movement of the pacing device while it is being delivered.

The delivery catheter or wire may be advanced to approximately 50-60 cm. This brings the pacing device into the superior vena cava/right atrium. From here, the pacing device may be released and, with venous flow, go into the right ventricle. The pacing device may be heavy enough that once released into the superior vena cava/right atrium it drops into the right ventricle. This should happen naturally with the flow blood. For example, the delivery catheter may be very soft and light (e.g., wire-like) with a heavy-weighted pacing device at the tip that may be advanced to the right ventricle without risk of perforation.

The exact deficits to which the catheter should be advanced may be measured externally on the patient to determine what would bring us close to the right atrium. For example, the pacing system may include markings indicative of how far the pacing device has been delivered.

Instead of a pellet-shaped pacing device, the pacing device may have a wire configuration, for example the pacing device may be a 0.014 wire. The wire may have an atraumatic tip, but is sufficiently stiff for pushability. The wire may be delivered or fixed using any of the techniques described below.

Although certain examples have been described herein as bipolar, any of the pacing systems described herein may be configured in a quadripolar configuration. For example, the system may be include two pacing devices (e.g., two pellets) with a first pacing device to pace the atrium and a second pacing device to pace the ventricle. The first pacing device may be connected to the second pacing device, for example by a wire. Each of the pacing devices may be affixed using any of the concepts described herein.

Various delivery techniques may be used from the antecubital approach. A short 6 French sheath may be used as the introducer. A 0.014 extra-support wire (e.g., mailman type) may be advanced until there is evidence of ventricular ectopy. This confirms that the wire is in the right ventricle. Use of the atraumatic wire enables the system to be delivered without visualization.

In some configurations, the wire itself may be the pacing device. In the pellet configuration, the pacing device may be advanced over this wire to approximately 50 cm, which brings the pacing device to the right atrium. The wire may be removed and the pacing device released allowing it to drop by virtue of its weight into the right atrium or guided to the desired position using any of the methods described below. The pacing device may be fixed via a magnetic field with an external magnet or using any of the other methods described below.

A long (45 cm) sheath may be advanced over the 0.014 wire. This allows positioning of the tip of the sheath in the subclavian/inferior vena cava. The pacing device may be loaded in the tip of a 4 French/5 French glide catheter and advanced 40-50 cm. From there, the pacing device may be released to flow to the right ventricle.

A balloon tipped 4 French/5 French catheter may be advanced into the pulmonary artery until there is evidence of ventricular ectopy to confirm the catheter has been advanced past the right ventricle. The balloon may have a rounded shape and be concentric with the catheter. After deflating the balloon, the pacing device may be released in the pulmonary artery. The catheter may be withdrawn back by 10 cm allowing the pacing device to fall into the right ventricle.

### Example 1

The 18 gauge Angiocath that is present in the antecubital vein may be used to introduce a 0.014 extra-support wire (such as a Mailman wire). The wire may be advanced while it is being spun. Once the wire reaches the right ventricle, ventricular ectopy will be noted on the patient’s heart monitor. The Angiocath may be removed and a 4 French sheath that has a 0.014 compatible dilator may be advanced. The sheath may have an estimated length at 50-60 cm long or long enough so that it reaches the superior vena cava.

The delivery catheter that has the pacing device attached to it may be advanced through the sheath. Once the tip of the delivery catheter is in the superior vena cava, the pacing device may be released. Because of the weight of the pacing device, the pacing device will advance into the right atrium and then the right ventricle (direction of blood flow). An external magnet may be used to ensure that it stays in contact with the right ventricular wall. Pacing cables may be attached in the pacing device may be used.

### Example 2

A balloon tipped catheter (e.g., a Swan-Ganz catheter) may be advanced until it is in the pulmonary artery. The pacing device may be released and the catheter pulled back allowing the pacing device to drop into the right ventricle. Fixation may be provided with an external magnet.

### Terminology

Although certain examples have been described herein with an antecubital approach, any of the pacing systems described herein may be introduced through the antecubital approach or other approaches. For example, in some embodiments, the pacing systems may be introduced through groin, jugular, or subclavian venous access.

As used herein, the relative terms “proximal” and “distal” shall be defined from the perspective of the delivery system. Thus, proximal refers to the direction of the handle and distal refers to the direction of the catheter tip.

The terms “comprising,” “including,” “having,” and the like are synonymous and are used inclusively, in an open-ended fashion, and do not exclude additional elements, features, acts, operations, and so forth. Also, the term “or” is used in its inclusive sense (and not in its exclusive sense) so that when used, for example, to connect a list of elements, the term “or” means one, some, or all of the elements in the list.

Although certain embodiments and examples have been described herein, it will be understood by those skilled in the art that many aspects of the delivery systems shown and described in the present disclosure may be differently combined and/or modified to form still further embodiments or acceptable examples. All such modifications and variations are intended to be included herein within the scope of this disclosure. A wide variety of designs and approaches are possible. No feature, structure, or step disclosed herein is essential or indispensable.

For purposes of this disclosure, certain aspects, advantages, and novel features are described herein. It is to be understood that not necessarily all such advantages may be achieved in accordance with any particular embodiment. Thus, for example, those skilled in the art will recognize that the disclosure may be embodied or carried out in a manner that achieves one advantage or a group of advantages as taught herein without necessarily achieving other advantages as may be taught or suggested herein.

Moreover, while illustrative embodiments have been described herein, the scope of any and all embodiments having equivalent elements, modifications, omissions, combinations (e.g., of aspects across various embodiments), adaptations and/or alterations as would be appreciated by those in the art based on the present disclosure. The limitations in the claims are to be interpreted broadly based on the language employed in the claims and not limited to the examples described in the present specification or during the prosecution of the application, which examples are to be construed as non-exclusive. Further, the actions of the disclosed processes and methods may be modified in any manner, including by reordering actions and/or inserting additional actions and/or deleting actions. It is intended, therefore, that the specification and examples be considered as illustrative only, with a true scope and spirit being indicated by the claims and their full scope of equivalents.

Conditional language used herein, such as, among others, “can,” “might,” “may,” “e.g.,” and the like, unless specifically stated otherwise, or otherwise understood within the context as used, is generally intended to convey that some embodiments include, while other embodiments do not include, certain features, elements, and/or states. Thus, such conditional language is not generally intended to imply that features, elements, blocks, and/or states are in any way required for one or more embodiments or that one or more embodiments necessarily include logic for deciding, with or without author input or prompting, whether these features, elements and/or states are included or are to be performed in any particular embodiment.

The methods disclosed herein may include certain actions taken by a “advancing a pacing catheter;” however, the methods can also include any third-party instruction of those actions, either expressly or by implication. For example, actions such as “advancing a pacing catheter” include “instructing advancing the pacing catheter.”

What is claimed is:

1. A method of delivering a pacing device to a heart of a patient, the method comprising:
advancing a pacing catheter to the heart in a first configuration;
shaping the pacing catheter by advancing a shaping instrument through a lumen of the pacing catheter and causing the pacing catheter to transition from the first configuration to a second configuration, the second configuration having a distal portion shaped to contact a wall of the heart and stabilize the pacing catheter in the heart; and
applying an electrical pulse from the distal portion of the pacing catheter to the wall of the heart.

2. The method of claim 1, further comprising, prior to advancing the shaping instrument, selecting the shaping instrument from a plurality of different-shaped shaping instruments.

3. The method of claim 1, further comprising, prior to advancing the shaping instrument, selecting the shaping instrument from a plurality of different-sized shaping instruments.

4. The method of claim 1, wherein shaping the pacing catheter occurs when a distal tip of the pacing catheter is in the right ventricle outflow tract.

5. The method of claim 1, further comprising applying the electrical pulse to the right ventricle outflow tract.

6. The method of claim 1, further comprising removing the shaping instrument from the lumen of the pacing catheter and causing the pacing catheter to transition from the second configuration to the first configuration; and removing the pacing catheter from the patient in the first configuration.

7. The method of claim 1, further comprising advancing the pacing catheter over a guidewire.

8. The method of claim 1, further comprising transmitting light from a sidewall of the pacing catheter while advancing the pacing catheter to the heart, the transmitted light being visible outside the patient.

9. The method of claim 1, further comprising inflating a balloon to carry the pacing catheter with blood flow.

10. The method of claim 1, further comprising deploying a fixation element to stabilize the pacing catheter in the heart.

11. The method of claim 10, further comprising deploying the fixation element in an atrium of the heart.

12. The method of claim 11, further comprising applying an electrical pulse from the fixation element to the atrium of the heart.

13. (canceled)

14. (canceled)

15. (canceled)

16. (canceled)

17. (canceled)

18. (canceled)

19. (canceled)

20. (canceled)

21. (canceled)

22. (canceled)

23. (canceled)

24. (canceled)

25. (canceled)

26. (canceled)

27. (canceled)

28. (canceled)

29. (canceled)

30. (canceled)

31. (canceled)

32. (canceled)

33. (canceled)

34. (canceled)

35. (canceled)

36. (canceled)

37. (canceled)

38. (canceled)

39. (canceled)

40. A method pacing the ventricles, the method comprising:
advancing a pacing catheter through the vasculature, the pacing catheter comprising:
a catheter body comprising an electrode region;
one or more light sources disposed on the catheter body;
a flotation balloon; and
a fixation element in the electrode region of the catheter body.
deploying the flotation balloon when light emitted from the one or more light sources is obstructed by the clavicle;
guiding a distal end of the catheter body to a right ventricular outflow tract using the flotation balloon; and
deploying the fixation element in the right ventricular outflow tract.

41. A method of delivering a pacing device to a heart of a patient, the method comprising:
introducing a pacing device through an access location to the antecubital vein;
advancing the pacing device to the heart without visualization; and
fixing the pacing device in the heart.

42. The method of claim 41, further comprising delivering IV fluids through the access location.

43. The method of claim 41, wherein advancing the pacing device comprises advancing a delivery catheter carrying the pacing device.

44. The method of claim 43, further comprising releasing the pacing device from the delivery catheter.

45. The method of claim 44, wherein releasing the pacing device comprises releasing the pacing device in a superior vena cava or right atrium of the heart.

46. The method of claim 41, wherein advancing the pacing device comprises advancing a wire having the pacing device.

47. The method of claim 41, further comprising introducing a second pacing device to the heart, the second pacing device connected to the first pacing device.

48. The method of claim 41, further comprising guiding the pacing device with an external magnet.

49. The method of claim 41, wherein fixing the pacing device comprises fixing the pacing device using one or more fixation components.

50. The method of claim 49, wherein fixing the pacing device comprises releasing the one or more fixation components after the pacing device reaches the pulmonary artery.
